(19) United States
(12) Patent Application Publication
(10) Pub. No.: US 2026/0167950 A1
(43) Pub. Date: Jun. 18, 2026

(54) PREPARATION METHOD AND APPLICATION FOR BIONIC ENZYME AEROGEL REACTOR

(71) Applicant: ZHEJIANG A&F UNIVERSITY, Hangzhou City (CN)

(72) Inventors: Weiwei HUAN, Hangzhou City (CN); Jiasheng Wu, Hangzhou City (CN); Lili Song, Hangzhou City (CN); Jie Li, Hangzhou City (CN); Huafeng Li, Hangzhou City (CN); Hongying Guo, Hangzhou City (CN); Jiajun Cui, Hangzhou City (CN); Yongqiang Gu, Hangzhou City (CN)

(21) Appl. No.: 19/414,555

(22) Filed: Dec. 10, 2025

(30) Foreign Application Priority Data
Dec. 16, 2024 (CN) 202411845352.7

(51) Int. Cl. C12N 11/14; C12N 9/18

(52) U.S. Cl. CPC C12N 11/14; C12N 9/18; C12Y 301/0102

(57) ABSTRACT

A preparation method and an application for a bionic enzyme aerogel reactor are provided. The preparation method includes following steps: step 1, first preparing titanium dioxide nanofibers (TiNFs) through solvothermal treatment; step 2, freeze-casting and vacuum-drying an aqueous dispersion including the TiNFs, chitosan methacryloyl (CSMA) and a crosslinking agent, to obtain a bionic CSMA/titanium dioxide nanofibers (C/TiNFs) aerogel reactor with vertically aligned channels; step 3, polymerizing and modifying glycidyl methacrylate (GMA), sulfobetaine methacrylate (SBMA) monomer and N,N′-methylenebisacrylamide (MBA) crosslinker on a skeleton of the bionic enzyme aerogel reactor; and step 4, covalently immobilizing tannase in the bionic enzyme aerogel reactor under mild conditions, thereby obtaining a bionic tannase-poly(GMA-SBMA-CSMA)/titanium dioxide nanofibers (pGSC/TiNFs) enzyme reactor.

### CROSS-REFERENCE TO RELATED APPLICATIONS

This application claims priority to Chinese Patent Application No. 202411845352.7, filed on Dec. 16, 2024, the contents of which are hereby incorporated by reference.

### TECHNICAL FIELD

The present disclosure relates to the technical field of preparation methods for bionic enzyme reactors, and in particular to a preparation method and an application for a bionic enzyme aerogel reactor.

### BACKGROUND

Enzymes possess characteristics such as high specificity, good biocompatibility, and efficient catalytic activity, and are widely used in fields including food production, biochemical detection, and pharmaceutical manufacturing. However, obstacles including insufficient stability, enzyme denaturation, and difficulties in separation and reuse limit efforts for the continuous conversion of reactants into products and for reducing production costs. In this regard, enzyme immobilization technology is considered an effective approach to overcome the aforementioned challenges. Currently, various enzyme immobilization methods have been developed, including physical adsorption, encapsulation, and chemical bonding. After immobilization, the stability of enzymes has been improved to a certain extent, but overall, it remains insufficient.

### SUMMARY

Aiming at the technical problems mentioned in the background art, the present disclosure provides a preparation method and an application for a bionic enzyme aerogel reactor.

The technical scheme adopted by the present disclosure is: a preparation method for a bionic enzyme aerogel reactor, specifically including the following steps:
step 1, first preparing titanium dioxide nanofibers (TiNFs) through solvothermal treatment;
step 2, freeze-casting and vacuum-drying an aqueous dispersion including the TiNFs, chitosan methacryloyl (CSMA) and a crosslinking agent, to obtain a bionic CSMA/titanium dioxide nanofibers (C/TiNFs) aerogel reactor with vertically aligned channels;
step 3, polymerizing and modifying glycidyl methacrylate (GMA) monomer, sulfobetaine methacrylate (SBMA) monomer and N,N′-methylenebisacrylamide (MBA) crosslinker on a skeleton of the bionic enzyme aerogel reactor; and
step 4, covalently immobilizing tannase in the bionic enzyme aerogel reactor under mild conditions, thereby obtaining a bionic tannase-poly(GMA-SBMA-CSMA)/titanium dioxide nanofibers (pGSC/TiNFs) enzyme reactor.

In an embodiment, the bionic tannase-pGSC/TiNFs enzyme reactor obtained in the step 4 is cylindrical.

In an embodiment, the bionic tannase-pGSC/TiNFs enzyme reactor obtained in the step 4 exhibits a three-dimensional hierarchical cell-like structure, with honeycomb pores in the structure.

In an embodiment, the honeycomb pores have a size of 20-100 micrometers, and mesh pore walls composed of nanofibers and polymers have a thickness of 0.2-2 micrometers.

In an embodiment, the bionic tannase-pGSC/TiNFs enzyme reactor also exhibits unidirectional, vertically aligned and interconnected channels in a longitudinal direction.

In an embodiment, an application of a bionic enzyme aerogel reactor applies the bionic tannase-pGSC/TiNFs enzyme reactor in bio-catalytic engineering.

The beneficial effects of the present disclosure are as follows. Compared with the prior art, the bionic tannase-pGSC/TiNFs enzyme reactor in the present disclosure is composed of TiNFs, CSMA, poly(glycidyl methacrylate) (PGMA), poly(sulfobetaine methacrylate) (PSBMA) and immobilized tannase, and has vertically aligned channels. Due to the reactor having characteristics of a honeycomb structure, rapid mass transfer, a comfortable micro-environment and a substrate enrichment effect from PSBMA modification, compared to enzyme reactors without PSBMA and free tannase, the bionic tannase-pGSC/TiNFs enzyme reactor exhibits enhanced pH and thermal tolerance, thermal stability, storage stability and reusability. The bionic tannase-pGSC/TiNFs enzyme reactor may act as a tissue cell reactor, and may continuously and efficiently convert tannin into gallic acid and glucose under continuous flow conditions, demonstrating application in biomass conversion.

### BRIEF DESCRIPTION OF THE DRAWINGS

FIG. 1A shows a schematic diagram of the preparation method for the bionic tannase-poly(GMA-SBMA-CSMA)/titanium dioxide nanofibers (pGSC/TiNFs) enzyme reactor with vertically aligned channels in the present disclosure.

FIG. 1B shows a schematic diagram of the application of the bionic tannase-pGSC/TiNFs enzyme reactor with vertically aligned channels in the continuous flow catalytic conversion of tannin.

FIG. 2A shows the bionic enzyme reactor.

FIG. 2B shows a scanning electron microscopy (SEM) image of the bionic tannase-pGSC/TiNFs enzyme reactor in the transverse direction.

FIG. 2C shows an SEM image of the bionic tannase-pGSC/TiNFs enzyme reactor in the transverse direction.

FIG. 2D shows an SEM image of the bionic tannase-pGSC/TiNFs enzyme reactor in the transverse direction.

FIG. 2E shows an SEM image of the bionic tannase-pGSC/TiNFs enzyme reactor in the longitudinal direction.

FIG. 2F shows the water contact angle diagram of the bionic tannase-pGSC/TiNFs enzyme reactor in the present disclosure.

FIG. 3A shows the Zeta-potential values of different samples after grinding in water in the present disclosure.

FIG. 3B shows the water flux of different samples under gravity without additional auxiliary pressure.

FIG. 3C shows the compressive stress-strain curves of different samples.

FIG. 3D shows the immobilization amount of tannase in different reactors in the present disclosure.

FIG. 4A shows the effects of immobilization time on the enzyme immobilization amount and activity of the bionic tannase-pGSC/TiNFs enzyme reactor in the present disclosure.

FIG. 4B shows the effects of tannase concentration on the enzyme immobilization amount and activity of the bionic tannase-pGSC/TiNFs enzyme reactor in the present disclosure.

FIG. 4C shows the effects of pH value on the enzyme immobilization amount and activity of the bionic tannase-pGSC/TiNFs enzyme reactor in the present disclosure.

FIG. 4D shows the effects of reaction temperature on the enzyme immobilization amount and activity of the bionic tannase-pGSC/TiNFs enzyme reactor in the present disclosure.

FIG. 5A shows the effect of pH value on the activity of immobilized and free tannase in the present disclosure.

FIG. 5B shows the effect of temperature on the activity of immobilized and free tannase in the present disclosure.

FIG. 5C shows the effect of storage time on the activity of immobilized and free tannase in the present disclosure.

FIG. 5D shows the effect of multiple cycles on the activity of immobilized and free tannase in the present disclosure.

FIG. 6A, FIG. 6B, FIG. 6C and FIG. 6D shows the continuous flow catalytic conversion of tannin using the bionic tannase-pGSC/TiNFs enzyme reactor in the present disclosure, where

FIG. 6A is a photograph of the test equipment.

FIG. 6B is the conversion efficiency at different flow rates.

FIG. 6C is the conversion efficiency over 10 consecutive cycles using 100 milliliters solution per cycle.

FIG. 6D is the conversion efficiency after continuous use for 15 days.

### DETAILED DESCRIPTION OF THE EMBODIMENTS

The technical schemes in the embodiments of the present disclosure will be clearly and completely described below in conjunction with the embodiments of the present disclosure. It is apparent that the described embodiments are only a part of the embodiments of the present disclosure, and not all of them. Based on the embodiments in the present disclosure, all other embodiments obtained by a person of ordinary skill in the art without making creative efforts fall within the scope of protection of the present disclosure.

In order to solve the problems existing in the background art, the present disclosure proposes the following technical scheme: a preparation method and an application for a bionic enzyme aerogel reactor. The preparation method includes the following steps.

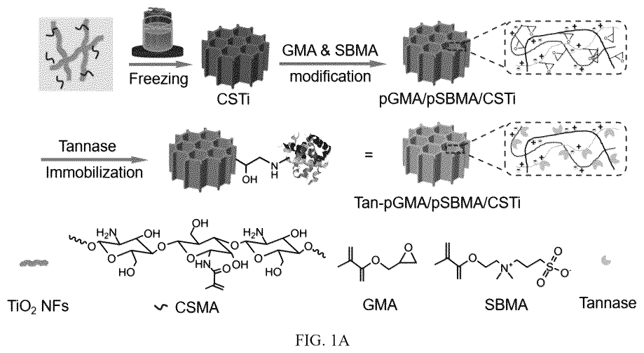

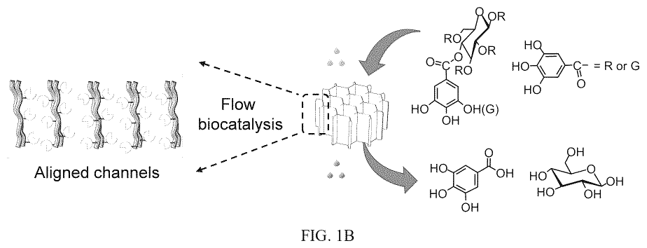

The preparation process is shown in FIG. 1A.
Step 1: titanium dioxide nanofibers (TiNFs) are first prepared through solvothermal treatment.
Step 2: an aqueous dispersion containing TiNFs, chitosan methacryloyl (CSMA) and a crosslinking agent is freeze-cast and vacuum-dried to obtain a bionic CSMA/titanium dioxide nanofibers (C/TiNFs) aerogel reactor with vertically aligned channels.
Step 3: glycidyl methacrylate (GMA) monomer, sulfobetaine methacrylate (SBMA) monomer and N,N′-methylenebisacrylamide (MBA) crosslinker are polymerized and modified on a skeleton of the bionic enzyme aerogel reactor.
Step 4: tannase is covalently immobilized in the bionic enzyme aerogel reactor under mild conditions, thereby obtaining a bionic tannase-poly(GMA-SBMA-CSMA)/titanium dioxide nanofibers (pGSC/TiNFs) enzyme reactor. The bionic tannase-pGSC/TiNFs enzyme reactor has high porosity and water flux. When a solution flows through the reactor, the reactant tannin may be efficiently converted into gallic acid and glucose (FIG. 1B).

Among them, the bionic tannase-pGSC/TiNFs enzyme reactor obtained in the step 4 is cylindrical. The bionic tannase-pGSC/TiNFs enzyme reactor exhibits a three-dimensional hierarchical cell-like structure, which features honeycomb pores in its structure. The honeycomb pores have a size of 20-100 micrometers, and the mesh pore walls are composed of nanofibers and polymers with a thickness of 0.2-2 micrometers. The bionic tannase-pGSC/TiNFs enzyme reactor also displays unidirectional, vertically aligned and interconnected channels in the longitudinal direction.

Further explanations are as follows.

The preparation method for the bionic C/TiNFs aerogel is as follows.

A 20.0 grams aqueous suspension of TiNFs (20 milligrams per gram), a 10.0 grams solution of CSMA (20 milligrams per gram), and 100 microliters of poly(ethylene glycol) diglycidyl ether are mixed and stirred for 20 minutes. The mixture is added into a cylindrical mold, and a directional freezing process is then performed using liquid nitrogen. The frozen sample is freeze-dried and heat-treated at 60 degrees Celsius for 4 hours to obtain the bionic C/TiNFs aerogel.

The synthesis method for the bionic tannase-poly(glycidyl methacrylate) (PGMA)/SBMA/CSMA/TiNFs (tannase-pGSCS/TiNFs) enzyme reactor is as follows.

400 milligrams of GMA, 12 milligrams of K₂S₂O₈, 2 milliliters of SBMA, and 12 milligrams of MBA are dissolved in 4 milliliters of deionized water. The resulting solution is dropped into the aforementioned bionic C/TiNFs aerogel, and the sample is heat-treated at 80 degrees Celsius for 1 hour to obtain the bionic pGSC/TiNFs reactor. The sample is washed with deionized water to remove residual chemicals. Subsequently, the pGSC/TiNFs reactor is added to a phosphate buffer (10 milliliters, pH 7.0) containing tannase at a concentration of 0.5 milligrams per milliliter. The mixture is incubated at a temperature of 4 degrees Celsius for 12 hours. The obtained tannase-immobilized enzyme reactor is washed several times with phosphate buffer (pH 7.0) and stored in a refrigerator.

Furthermore, the determination methods for the enzyme activity, enzyme immobilization amount, and conversion efficiency of the bionic tannase-pGSCS/TiNFs enzyme reactor in continuous flow mode are as follows.

The tannase activity is investigated by measuring the amount of gallic acid produced from methyl gallate. The amount of immobilized enzyme in the bionic tannase-pGSCS/TiNFs enzyme reactor is determined using the Bradford method. The continuous flow catalytic conversion test is conducted in a conventional chromatography column. A bionic tannase-pGSCS/TiNFs enzyme reactor with a diameter of 26 millimeters and a height of 26 millimeters is placed at the bottom of the column. An aqueous solution containing tannin (1 milligram per milliliter) is injected onto the top surface of the bionic tannase-pGSCS/TiNFs enzyme reactor using a peristaltic pump. The initial solution and the filtered solution after catalytic treatment are collected. The initial solution and the filtered solution after catalytic treatment are collected and analyzed by ultraviolet-visible absorption spectroscopy.

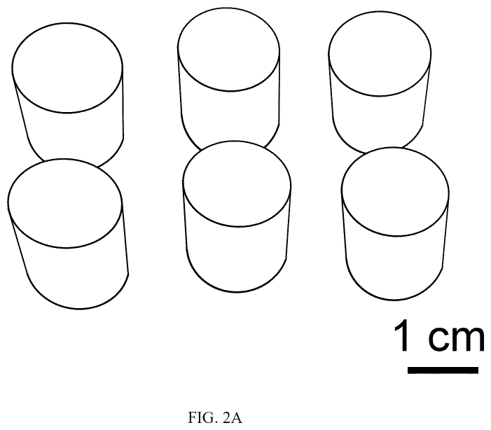

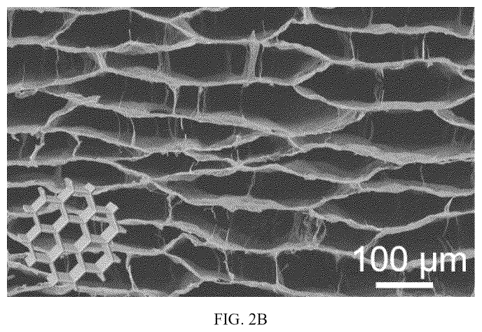

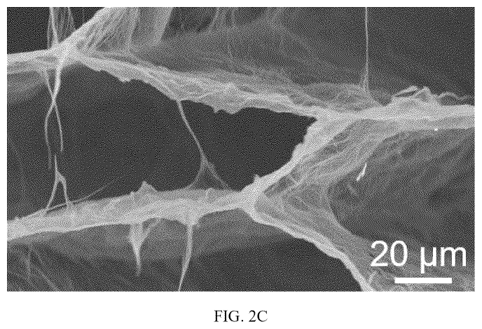

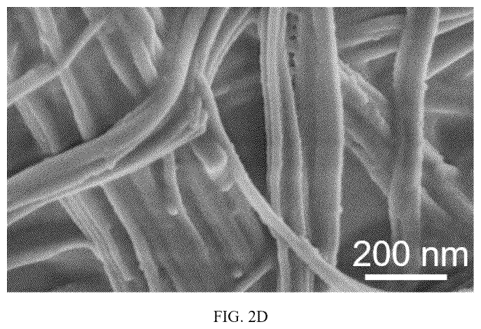

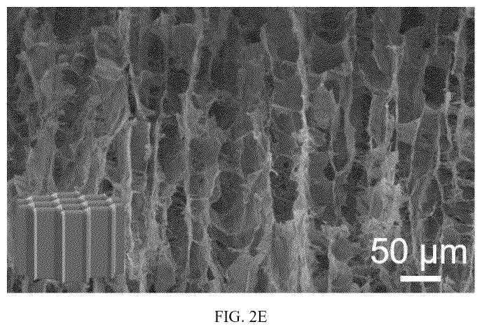

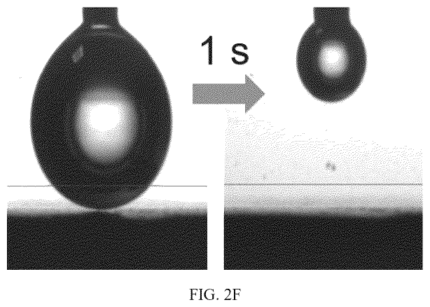

In summary, the surface and internal morphology of the bionic tannase-pGSC/TiNFs enzyme reactor are observed. The bionic tannase-pGSC/TiNFs enzyme reactor is cylindrical and may be easily prepared in a cylindrical mold (FIG. 2A).

FIG. 2B, FIG. 2C and FIG. 2D show the scanning electron microscopy (SEM) images in the transverse direction. The bionic tannase-pGSC/TiNFs enzyme reactor exhibits a three-dimensional hierarchical cell-like structure, and its structure resembles the honeycomb pores found in natural wood.

Among them, the honeycomb pores have a size of 20-100 micrometers, and the mesh pore walls are composed of nanofibers and polymers with a thickness of 0.2-2 micrometers. Furthermore, the bionic tannase-pGSC/TiNFs enzyme reactor exhibits unidirectional, vertically aligned and interconnected channels in the longitudinal direction (FIG. 2E). This result is attributed to the continuous growth of ice crystals in the vertical direction.

Additionally, the bionic tannase-pGSC/TiNFs enzyme reactor is hydrophilic, and water droplets may be completely absorbed within 1 second (as shown in FIG. 2F).

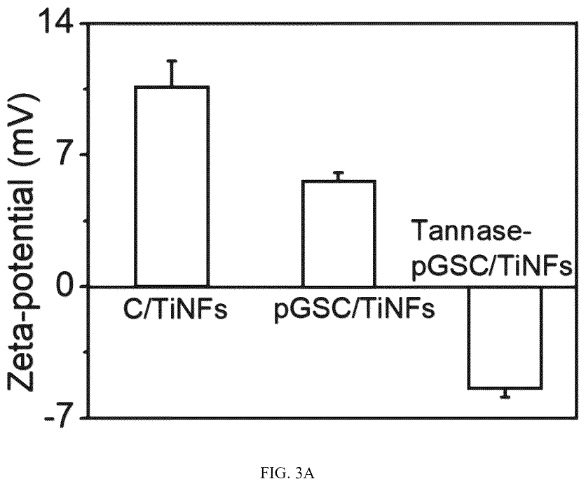

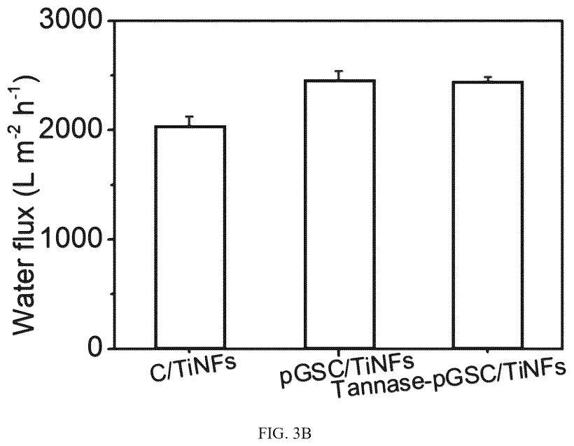

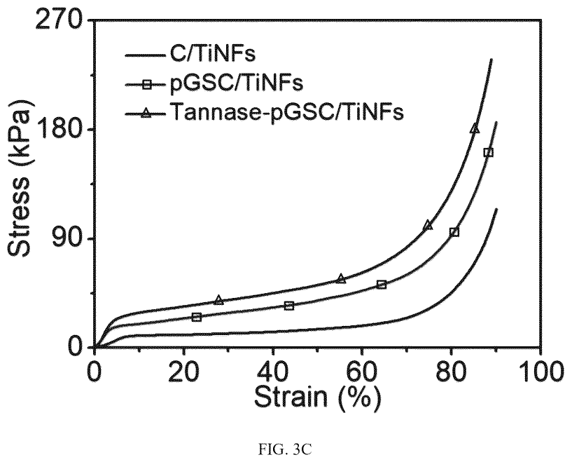

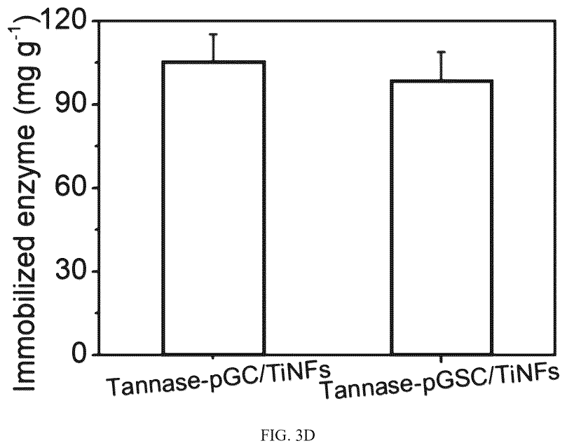

In further design, the surface ζ potential of the prepared samples is investigated. As shown in FIG. 3A, the C/TiNFs sample prepared from TiNFs and CSMA has a positive potential of +10.5 millivolts, after subsequent modification with PGMA and poly(sulfobetaine methacrylate) (PSBMA), the surface potential is reduced to approximately +5.60 millivolts, furthermore, the tannase-pGSC/TiNFs enzyme reactor has a negative potential of −5.31 millivolts, indicating that tannase is successfully immobilized. Benefiting from excellent water wettability and high porosity (99.2%), aqueous solutions may rapidly flow through the reactor under gravity without requiring additional auxiliary pressure.

Among them, the water fluxes of the bionic C/TiNFs, pGSC/TiNFs, and tannase-pGSC/TiNFs enzyme reactors with a thickness of 15 millimeters are determined to be 2023, 2454, and 2432 liters per square meter per hour, respectively (FIG. 3B). The high water flux and low physical flow resistance contribute to rapid reactant-product exchange in continuous flow catalytic reactions.

Additionally, FIG. 3C shows the compressive stress-strain curves of the three samples. Among them, the compressive stress at 70% strain for the bionic tannase-pGSC/TiNFs enzyme reactor (83.3 kilopascals) is higher than that for pGSC/TiNFs (60.4 kilopascals) and C/TiNFs (24.9 kilopascals). The compressive stress at 70% strain for the bionic tannase-pGSC/TiNFs enzyme reactor remains higher than that of the bionic porous materials with similar structures reported in the literature.

Among them, the enzyme immobilization amount on the bionic reactor with PSBMA modification is determined. As shown in FIG. 3D, the amount of tannase immobilized on tannase-pGSC/TiNFs (98.4 milligrams per gram) is slightly lower than that on tannase-poly(GMA-CSMA)/titanium dioxide nanofibers (pGC/TiNFs) (104.5 milligrams per gram).

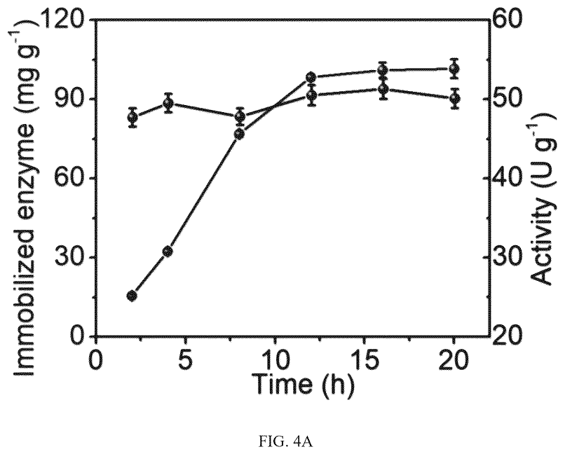

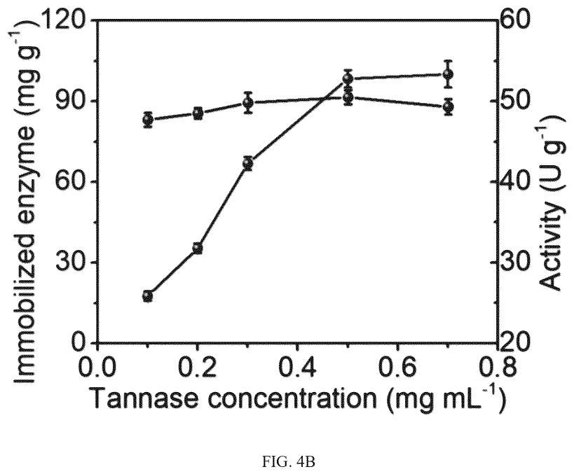

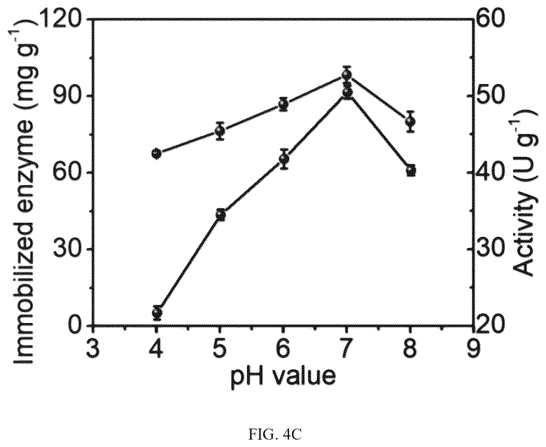

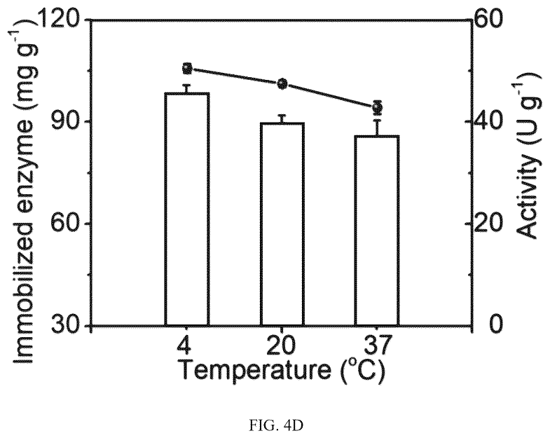

Among them, the present disclosure investigates the effects of tannase concentration, reaction time, pH, and temperature on the immobilized enzyme binding amount and enzyme activity. The results show that the optimal immobilization efficiency is achieved under the conditions of a tannase concentration of 0.5 milligram per milliliter, a pH of 7.0, a reaction temperature of 4 degrees Celsius, and a reaction time of 12 hours (FIG. 4A, FIG. 4B, FIG. 4C and FIG. 4D). The data indicates that the enzyme binding amount, immobilization efficiency, and enzyme activity are 98.4 milligrams per gram, 84.2%, and 50.5 units per gram, respectively.

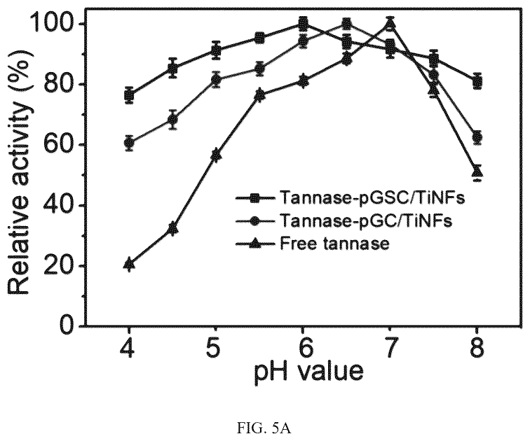

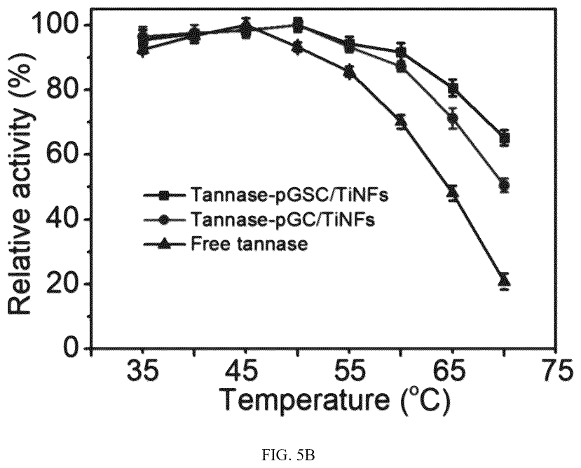

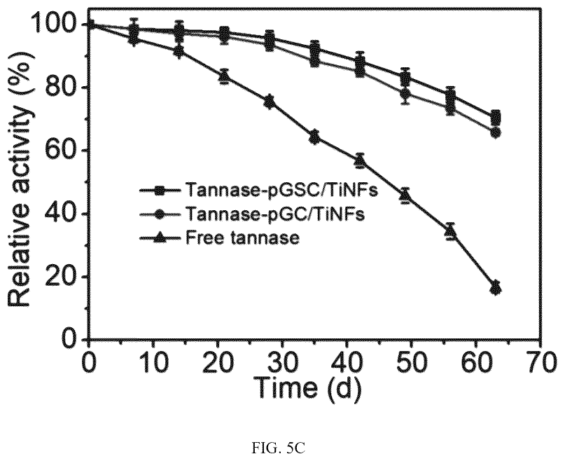

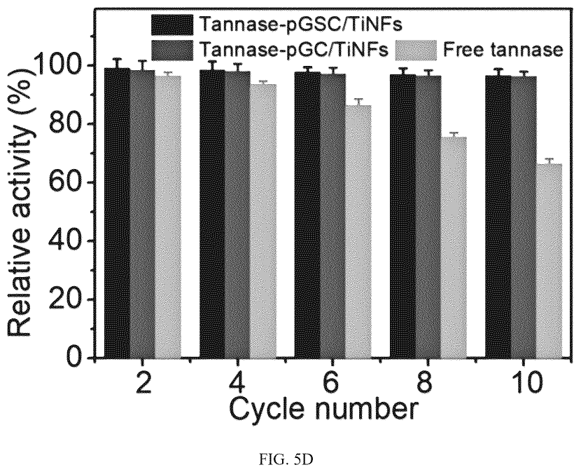

Furthermore, the effects of pH, temperature, storage time, and cycle number on the activities of tannase-pGC/TiNFs, tannase-pGSC/TiNFs, and free tannase are also investigated. As shown in FIG. 5A, the optimal pH for free tannase is 7.0, while tannase-pGC/TiNFs and tannase-pGSC/TiNFs exhibit maximum activity at pH 6.5 and pH 6.0, respectively. Moreover, within the pH range of 5.0 to 6.0, tannase-pGC/TiNFs and tannase-pGSC/TiNFs maintain activities of 81.6-94.3% and 91.3-100%, respectively. In contrast, free tannase remains in a lower range of 56.7-81.2%. At a pH of 8.0, the residual activities of both tannase-pGC/TiNFs (62.5%) and tannase-pGSC/TiNFs (81.2%) are higher than that of free tannase (50.8%).

The results indicate that the immobilized tannase possesses higher stability over a wider pH range compared to free tannase. This phenomenon is attributed to the modified polymers PGMA and PSBMA, which impart a buffering micro-environment and excellent protective effects to tannase-pGC/TiNFs and tannase-pGSC/TiNFs, providing them with a shielding effect.

Among them, the stability of immobilized tannase and free tannase at different temperatures is also investigated. As shown in FIG. 5B, free tannase exhibits maximum activity at 45 degrees Celsius, while tannase-pGC/TiNFs and tannase-pGSC/TiNFs reach their optimum activity at 50 degrees Celsius. Furthermore, an increase in temperature from 45 degrees Celsius to 65 degrees Celsius causes the activities of tannase-pGC/TiNFs and free tannase to sharply decrease to 71.2% and 48.1%, respectively, whereas tannase-pGSC/TiNFs maintains a relatively high activity of 80.6%. Even at a high temperature of 70 degrees Celsius, the remaining activity of tannase-pGSC/TiNFs is still superior to that of tannase-pGC/TiNFs and free tannase. These results are due to the covalent bonds between tannase and the solid carriers in pGC/TiNFs and pGSC/TiNFs, which enhance the molecular rigidity and thermal stability of the enzyme protein.

The storage stabilities of free and immobilized tannase are determined by incubating free tannase in a buffer solution at pH=7.0 and incubating the immobilized enzymes in a buffer solution at pH=6.5 in 63 days. Free tannase retains 17.8% of its initial activity, while tannase-pGC/TiNFs and tannase-pGSC/TiNFs maintain 64.6% and 70.5% of their enzyme activities, respectively (FIG. 5C). Furthermore, after 10 consecutive cycles are implemented, tannase-pGC/TiNFs and tannase-pGSC/TiNFs show high activity retention rates of 96.3% and 96.5%, but the relative activity of free tannase is 66.5% (FIG. 5D). These results indicate that the protein structure of tannase is stable after it is covalently immobilized on the designed carriers.

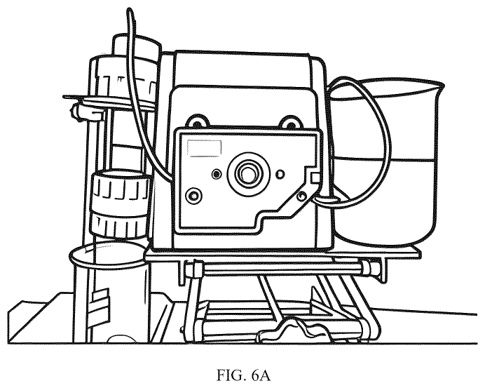

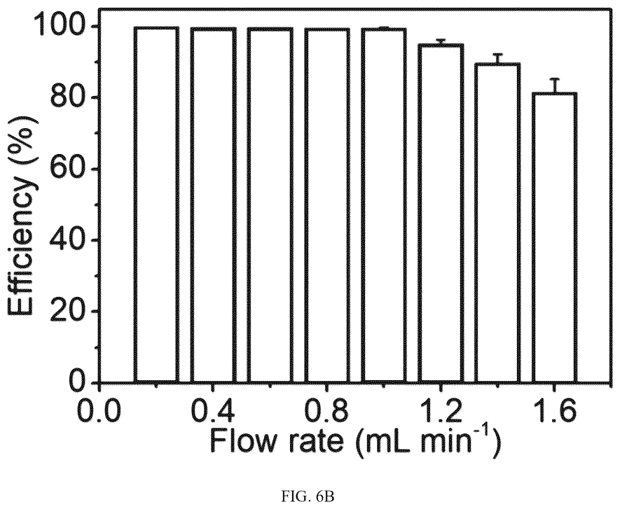

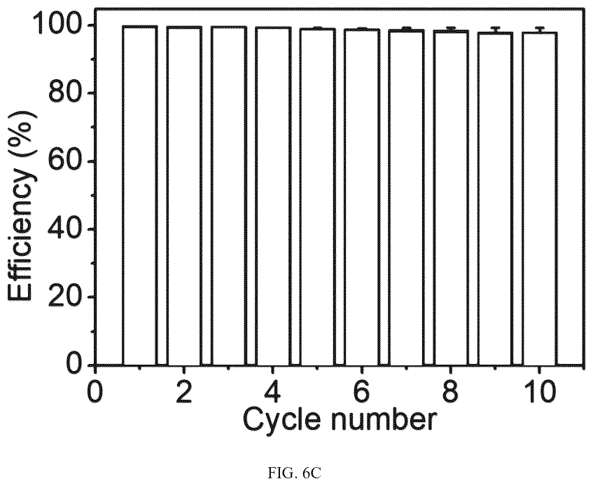

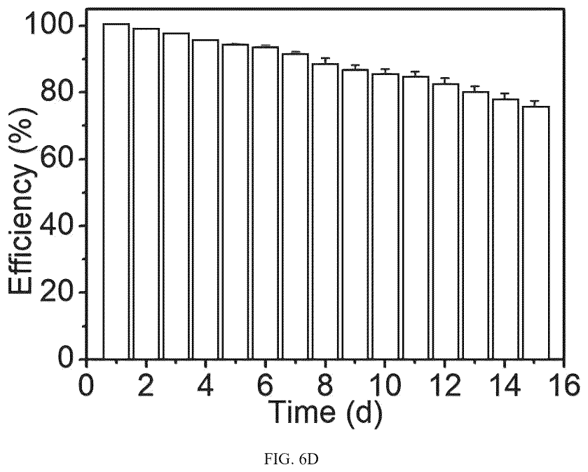

Among them, furthermore, the continuous flow catalytic conversion of tannin to gallic acid and glucose is also evaluated. As shown in FIG. 6A, the bionic tannase-pGSC/TiNFs enzyme reactor is fixed in the device, and an aqueous solution containing the reactant is pumped onto the top surface of the bionic tannase-pGSC/TiNFs enzyme reactor. The catalytic reaction is completed as the aqueous solution flows through the tannase-pGSC/TiNFs enzyme reactor. The effect of flow rate on the conversion efficiency is also studied. As shown in FIG. 6B, when the flow rate is lower than 1.0 milligram per milliliter, the conversion efficiency exceeds 99.6%. When the flow rate is further increased, the efficiency gradually decreases. Excessively high flow rates may lead to insufficient contact between the immobilized tannase and the reactants.

The recyclability and long-term stability of the bionic tannase-pGSC/TiNFs enzyme reactor are investigated. The flow catalytic conversion of tannin is continuously repeated for 10 cycles in the presence of the bionic tannase-pGSC/TiNFs enzyme reactor. The conversion efficiency slightly decreases as the number of cycles increases (FIG. 6C). The bionic tannase-pGSC/TiNFs enzyme reactor may still maintain 98.1% efficiency after 10 cycles, showing good recyclability. Furthermore, the bionic tannase-pGSC/TiNFs enzyme reactor is operated continuously for 15 days, and the conversion efficiency may still be maintained at 82.4% (FIG. 6D). Therefore, the prepared bionic tannase-pGSC/TiNFs enzyme reactor possesses high conversion efficiency, good recyclability, and long-term stability in the continuous bioconversion of tannin.

In summary, the bionic tannase-pGSC/TiNFs enzyme reactor in the present disclosure is composed of TiNFs, CSMA, PGMA, PSBMA and immobilized tannase, and has vertically aligned channels. Because the reactor possesses characteristics of a honeycomb structure, rapid mass transfer, a comfortable micro-environment and a substrate enrichment effect from PSBMA modification, compared to enzyme reactors without PSBMA and free tannase, the bionic tannase-pGSC/TiNFs enzyme reactor exhibits enhanced pH and thermal tolerance, thermal stability, storage stability and reusability. The modified PSBMA provides a buffering micro-environment and excellent protective effects, which contribute to high enzyme catalytic stability. Importantly, the bionic tannase-pGSC/TiNFs enzyme reactor may act as a tissue cell reactor, and may continuously and efficiently convert tannin into gallic acid and glucose under continuous flow conditions, demonstrating application potential in biomass conversion.

Although the embodiments of the present disclosure have been shown and described, the scope of the present disclosure is defined by the appended claims and their equivalents for a person of ordinary skill in the art.

What is claimed is:

1. A preparation method for a bionic enzyme aerogel reactor, comprising following steps:
step 1, first preparing titanium dioxide nanofibers (TiNFs) through solvothermal treatment;
step 2, freeze-casting and vacuum-drying an aqueous dispersion comprising the TiNFs, chitosan methacryloyl (CSMA) and a crosslinking agent, to obtain a bionic CSMA/titanium dioxide nanofibers (C/TiNFs) aerogel reactor with vertically aligned channels;
step 3, polymerizing and modifying glycidyl methacrylate (GMA) monomer, sulfobetaine methacrylate (SBMA) monomer and N,N′-methylenebisacrylamide (MBA) crosslinker on a skeleton of the bionic enzyme aerogel reactor; and
step 4, covalently immobilizing tannase in the bionic enzyme aerogel reactor under mild conditions, thereby obtaining a bionic tannase-poly(GMA-SBMA-CSMA)/titanium dioxide nanofibers (pGSC/TiNFs) enzyme reactor.

2. The preparation method for the bionic enzyme aerogel reactor according to claim 1, wherein the bionic tannase-pGSC/TiNFs enzyme reactor obtained in the step 4 is cylindrical.

3. The preparation method for the bionic enzyme aerogel reactor according to claim 1, wherein the bionic tannase-pGSC/TiNFs enzyme reactor obtained in the step 4 exhibits a three-dimensional hierarchical cell-like structure, with honeycomb pores in the three-dimensional hierarchical cell-like structure.

4. The preparation method for the bionic enzyme aerogel reactor according to claim 3, wherein the honeycomb pores have a size of 20-100 micrometers, and mesh pore walls composed of nanofibers and polymers have a thickness of 0.2-2 micrometers.

5. The preparation method for the bionic enzyme aerogel reactor according to claim 1, wherein the bionic tannase-pGSC/TiNFs enzyme reactor exhibits unidirectional, vertically aligned and interconnected channels in a longitudinal direction.
